(19) United States
(12) Patent Application Publication
(10) Pub. No.: US 2025/0295817 A1
(43) Pub. Date: Sep. 25, 2025

(54) MRI COMPOSITION AND ITS USE IN NONFASTING SUBJECTS

(71) Applicant: Ascelia Pharma AB, Malmö (SE)

(72) Inventors: Magnus Olafsson Corfitzen, Malmö (SE); Andreas Norlin, Malmö (SE)

(21) Appl. No.: 18/861,742

(22) PCT Filed: May 4, 2023
(86) PCT No.: PCT/EP2023/061741

§ 371 Date: Oct. 30, 2024

(30) Foreign Application Priority Data
May 5, 2022 (EP) 22171769.7

(51) Int. Cl. A61K 49/06; A61K 47/10; A61K 47/18

(52) U.S. Cl. CPC A61K 49/06; A61K 47/10; A61K 47/183

(57) ABSTRACT

The present invention relates to an oral Magnetic Resonance Imaging (MRI) composition and its use in diagnosis in vivo of liver metastases and/or primary liver cancer in subjects having consumed a predefined meal.

### TECHNICAL FIELD

The present invention relates to an oral Magnetic Resonance Imaging (MRI) composition and its use in diagnosis in vivo of liver metastases and/or primary liver cancer in subjects having consumed a predefined meal.

### BACKGROUND

Orviglance is an oral liver imaging drug (i.e. a liver specific contrast agent) for detection and visualization of focal liver lesions, using Magnetic Resonance Imaging (“MRI”) in subjects where use of the current gold standard gadolinium-based contrast agents (“GBCAs”) may be medically inadvisable or cannot be administered. MRI is a medical imaging technique used in radiology to form pictures of the anatomy and the physiological processes of the body. MRI scanners use strong magnetic fields, magnetic field gradients, and radio waves to generate images of the organs in the body.

A study by Leander et al (Investigative Radiology, 45, 2010) has previously shown that administration of an oral manganese based MRI composition to a nonfasting subject followed by MRI led to very low signal enhancement of the liver and the subject was excluded from further efficacy analyses.

Hence, fasting prior to administration of Orviglance as well as other contrast agents is routine procedure to ensure that the imaging quality is not affected by interactions with the various food components impacting absorption of the contrast agents and/or directly affecting the imaging properties of manganese (II) ions or complexes thereof.

### SUMMARY

The present inventors have confirmed that subjects' consumption of a full meal prior to administration of Orviglance results in a significantly inferior image quality than observed for fasting subjects. Long periods of fasting (>8 h) are generally undesirable and may limit the willingness to use Orviglance, and for some subjects, fasting for prolonged periods of time can impose a health risk or significant inconvenience.

Surprisingly, the present inventors have now demonstrated that a meal with a calorie content of a predefined value or below can be consumed prior to administration of Orviglance to obtain an imaging quality which is at least as high as obtained for fasting subjects. These findings support the diagnostic potential of Orviglance, in particular for subjects where fasting is significantly inconvenient or imposes a health risk.

In a first aspect, an MRI composition is provided comprising, separately or together, a physiologically acceptable manganese (II) compound, a proteinogenic amino acid and/or a vitamin D, and one or more water-soluble excipients; wherein the MRI composition is for use in the diagnosis in vivo of liver metastases and/or primary liver cancer in a subject; wherein the subject has consumed a meal from 0 to 8 hours prior to or up to about 2 hours subsequent to administration of the MRI composition and wherein the calorie content of the meal is about a predefined value or below.

In a second aspect, an MRI composition is provided comprising, separately or together, a physiologically acceptable manganese (II) compound, a proteinogenic amino acid and/or a vitamin D, and one or more water-soluble excipients; wherein the MRI composition is for use in the diagnosis in vivo of liver metastases and/or primary liver cancer in a subject; wherein the subject has consumed a meal from 0 to 8 hours prior to or up to about 2 hours subsequent to administration of the MRI composition and wherein the calorie content of the meal is about 500 kcal or below.

In a third aspect, a method for Magnetic Resonance Imaging (MRI) of a subject is provided comprising, administering a composition comprising separately or together, a physiologically acceptable manganese (II) compound, a proteinogenic amino acid and/or a vitamin D, and one or more water-soluble excipients to the subject; and performing MRI on said subject; wherein the subject has consumed a meal from 0 to 8 hours prior to or up to about 2 hours subsequent to administration of the MRI composition, and wherein the calorie content of the meal is about 500 kcal or below.

In a fourth aspect, a method of reducing or preventing nausea, diarrhea, and/or headaches in a subject receiving an MRI composition is provided comprising, separately or together, a physiologically acceptable manganese (II) compound, a proteinogenic amino acid and/or a vitamin D, and one or more water-soluble excipients; the method comprising:
i) administering a meal to the subject; and
ii) administering the MRI composition to the subject;
wherein the meal is administered from 0 to 8 hours prior to or up to about 2 hours subsequent to administering the MRI composition.

### Definitions

The term “administration” as used herein refers to the act of the MRI composition entering the body of a subject. Hence, time calculations prior to, or subsequent to administration are calculated in relation to the point in time where the MRI composition enters the body of a subject.

The term “meal”, “predefined meal”, “snack” or “light meal” as used herein interchangeably refer to a food serving having a calorie content of about a predefined value or below, such as about 500 kcal or below. The predefined value refers to a calorie content which is significantly below the calorie content of a full meal having a calorie content of about 900 kcal or higher.

The term “water-soluble” is used herein to describe a compound that dissolve in water at room temperature, such as about 25° C. The extent of water-solubility ranges widely, from infinitely soluble (without limit) (fully miscible) such as ethanol in water, to poorly soluble, such as silver chloride in water. The term insoluble is often applied to poorly or very poorly soluble compounds.

The term “water-insoluble” is used herein to describe a compound that does not dissolve in water at room temperature, such as about 25° C.

A number of other descriptive terms are also used to quantify the extent of solubility for a given solute, i.e. the compound subject to solution:

| | Mass parts of water |
|---|---|
| | required to dissolve 1 |
| Term | mass part of solute |
| Very soluble | <1 |
| Freely soluble | 1 to 10 |
| Soluble | >10 to 30 |
| Sparingly soluble | >30 to 100 |
| Slightly soluble | >100 to 1000 |
| Very slightly soluble | >1000 to 10,000 |
| Practically insoluble or insoluble | ≥10,000 |

The water-solubility of a given solute typically depends on temperature. Depending on the nature of the solute the solubility may increase or decrease with temperature. For most solids and liquids, their solubility in water increases with temperature.

The term “hygroscopic” is used herein to describe a compound that sorbs water, either by absorption, adsorption, or a combination of the two processes at between 40 and 60% relative humidity (rH) to any significant extent, such as to the extent wherein the mass and/or volume of said compound increases by about 5% or more within a period of time, such as after about 1 hour.

The term “non-hygroscopic water soluble” is used herein to describe a compound that is not hygroscopic in accordance with the definition herein, and at the same time is water-soluble according to the definition herein.

The term “absorption promoter” is used herein interchangeably with the term “uptake promoter” which refers to a compound capable of enhancing manganese transport across the membranes of the gastrointestinal tract. These compounds are well known in the art from e.g. WO 96/05867 and comprise physiologically tolerable reducing compounds containing an α-hydroxy ketone group, a physiologically tolerable acid containing α- and/or β-hydroxy or amino groups, or a salt thereof, and/or vitamin D.

### DESCRIPTION OF DRAWINGS

FIG. 1: Overview of study design from Example 1 with two periods of fasting and two groups of subjects receiving a full meal or a snack prior to dosing with Orviglance.

FIG. 2: Relative change in MRI signal intensity (SI %) from liver imaging at 1, 4, 8 and 24 hours post-dose for fasting subjects (black lines) compared to the same subjects after receiving a full meal (dotted lines). The SI % obtained from imaging of the subjects after a full meal is significantly lower than after the fasting period. The contents of the full meal is further described in Table 1.

FIG. 3: Relative change in MRI signal intensity (SI %) from liver imaging at 1, 4, 8 and 24 hours post-dose for fasting subjects (black lines) compared to the same subjects after receiving a snack (dotted lines). The SI % obtained from imaging of the subject after the snack is surprisingly at least as good as after fasting. The contents of the snack is further described in Table 2.

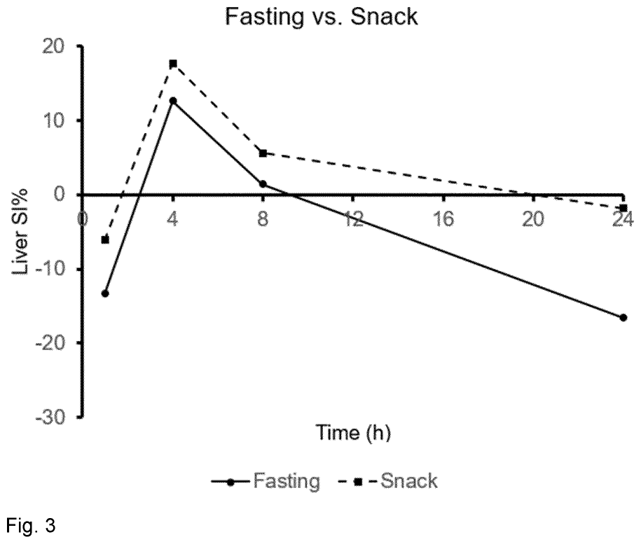

### DETAILED DESCRIPTION

In one embodiment, an MRI composition is provided comprising, separately or together, a physiologically acceptable manganese (II) compound, a proteinogenic amino acid and/or a vitamin D, and optionally one or more water-soluble excipients; wherein the MRI composition is for use in the diagnosis in vivo of liver metastases and/or primary liver cancer in a subject; wherein the subject has consumed a meal within 8 hours prior to or up to about 2 hours subsequent to administration of the MRI composition and wherein the calorie content of the meal is about 500 kcal or below.

In one embodiment, use of the MRI composition reduces or prevents nausea, diarrhea, and/or headaches in the subject as compared to a fasting subject that has not consumed food for 3 hours or more prior to administration of the MRI composition. That is to say in one embodiment the subject has reduced nausea, and/or diarrhea, and/or headaches as compared to a fasting subject having been administered said MRI composition.

In one embodiment, the subject has not been fasting for 3 or more hours prior to the administration of the MRI composition.

### Subjects Negatively Affected by Fasting

In addition to fasting being unpleasant or inconvenient, some subjects may be negatively affected by fasting for health reasons. For these subjects, the MRI composition and its use described herein are particularly well suited. Hence, in one embodiment, MRI composition is provided for use with a subject which is negatively affected by not eating for more than 8 hours.

In one embodiment, the subject suffers from a disease where not eating for more than 8 hours imposes a health risk to the subject. Hence, in some embodiments, the present disclosure provides an MRI composition for use with a subject which has not been eating for more than 8 hours prior to administration of the MRI composition.

In one embodiment, the subject has one or more of:
a. diabetes, such as diabetes type-I or diabetes type-II;
b. a Body Mass Index (BMI) of 20 or below;
c. an age of 60 years or above; and/or
d. hyperthyroidism.

### Timing

In one embodiment, the subject has consumed the meal from about 0 to 8 hours prior to or up to about 2 hours subsequent to administration of the MRI composition, such as from about 0 to 1 hour, such as from about 1 to 2 hours, such as from about 2 to 3 hours, such as from about 3 to 4 hours, such as from about 4 to 5 hours, such as from about 5 to 6 hours, such as from about 6 to 7 hours, such as from about 7 to 8 hours prior to administration of the MRI composition. In one embodiment, the subject has consumed the meal from up to about 30 minutes subsequent to, such as up to about 1 hour subsequent to, such as up to about 2 hours subsequent to administration of the MRI composition.

In one embodiment, the subject has consumed the meal within about 1 hour prior to administration of the MRI composition, such as within about 45 minutes, such as within about 30 minutes prior to administration of the MRI composition.

In one embodiment, the subject undergoes MRI from about 30 minutes to about 8 hours after administration of the MRI composition, such as from 2 hours to 3 hours, such as from 3 hours to 4 hours, such as from 4 hours to 5 hours, such as from 5 hours to 6 hours, such as from 6 hours to 7 hours, such as from 7 hours to 8 hours after administration of the MRI composition. In one particular embodiment, the subject undergoes MRI about 4 hours after administration of the MRI composition.

In one embodiment, the MRI composition is provided comprising, separately or together, a physiologically acceptable manganese (II) compound, a proteinogenic amino acid and/or a vitamin D, and optionally one or more water-soluble excipients; wherein the MRI composition is for use in the diagnosis in vivo of liver metastases and/or primary liver cancer in a subject; wherein the subject has consumed a meal within 2 hours prior to administration of the MRI composition and wherein the calorie content of the meal is about 500 kcal or below, and wherein the subject undergoes MRI about from 30 minutes to 6 hours, such as 4 hours after administration of the MRI composition.

Meal Having a Calorie Content about 500 Kcal or Below

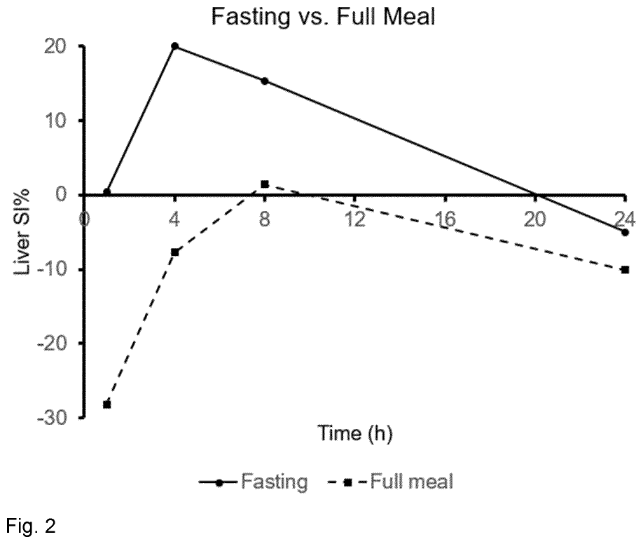

Some meals contain too many calories, such as more than 500 kcal, such as more than 600 kcal, such as more than 700 kcal, such as more than 800 kcal, such as more than 900 kcal and significantly interfere with the signal intensity enhancement during MRI as is demonstrated in Example 1 and as shown on FIG. 2. Such negative food effect on imaging was also recognized in the art, by Leander et al (Investigative Radiology, 45, 2010). An example of a meal, referred to herein as a full mean, which significantly negatively impact the MRI quality is shown in Table 1.

TABLE 1
Contents of a full meal.

| | | | Qty. Of | | Calorie | CHO | Fats | Proteins |
|---|---|---|---|---|---|---|---|---|
| Food Items | Servings | Ingredients | Ingredients | | (Kcal) | (gms) | (gms) | (gms) |
| Toast | 1 no | Bread | 1 | Slice | 61.25 | 12.98 | 0.18 | 1.95 |
| | | Butter | 10 | gms | 72.90 | 0.00 | 8.10 | 0.00 |
| *Chana | 60 gms | Chana | 20 | gms | 72.00 | 12.18 | 1.06 | 3.42 |
| Chat | | Onions | 5 | gms | 2.95 | 0.63 | 0.01 | 0.09 |
| | | Peanuts | 10 | gms | 56.7 | 2.61 | 4.01 | 2.53 |
| | | Oil | 2 | ml | 18.00 | 0.00 | 2.00 | 0.00 |
| Vegetable | 1 serving / | Potatoes | 50 | gms | 48.50 | 11.30 | 0.05 | 0.80 |
| Cutlets | 2 nos | Cheese | 40 | gms | 139.20 | 2.52 | 10.04 | 9.64 |
| (Approx 130 | | Onions | 5 | gms | 2.95 | 0.63 | 0.01 | 0.09 |
| gms) | | Bread | ¼ | Slice | 15.31 | 3.24 | 0.04 | 0.49 |
| | | Oil | 10 | ml | 90.00 | 0.00 | 10.00 | 0.00 |
| | | Paneer | 50 | gms | 146.00 | 3.95 | 11.50 | 6.70 |
| Milk^{#} | 1 serving | Milk | 200 | ml | 192.00 | 10.00 | 13.00 | 8.60 |
| | | Sugar | 5 | gms | 19.90 | 4.97 | 0.00 | 0.01 |

| | | | | |
|---|---|---|---|---|
| TOTAL | 937.66 | 65.01 | 60.00 | 34.32 |

| | | | |
|---|---|---|---|
| NUTRIENT CALORIES | 260.04 | 540.00 | 137.28 |
| NUTRIENT CALORIES AS % TOTAL | 27.73% | 57.59% | 14.64% |

In one embodiment, the meal according to the present disclosure having a calorie content about 500 kcal or below is characterized as a snack or a light meal. An exemplary overview of the contents of the meal having a calorie content about 500 kcal or below is provided in Table 2.

TABLE 2
Contents of a snack.

| Food | | | | Qty. Of | | Calorie | CHO | Fats | Proteins |
|---|---|---|---|---|---|---|---|---|---|
| Items | Servings | | Ingredients | Ingredients | | (Kcal) | (gm) | (gm) | (gm) |
| Toast | 1 | slice | Bread | 1 | slice | 61.25 | 12.98 | 0.18 | 1.95 |
| | | | Butter | 10 | gms | 72.90 | 0.00 | 8.10 | 0.00 |
| Sweet | 200 | ml | Sweet | 50 | gm | 13.64 | 2.59 | 0.1 | 0.38 |
| Lime | | | Lime Pulp | | | | | | |
| Juice | | | sugar | 15 | gm | 59.7 | 14.91 | 0.00 | 0.02 |

| | | | | |
|---|---|---|---|---|
| Total | 207.49 | 30.48 | 8.38 | 2.35 |

| | | | |
|---|---|---|---|
| Nutrients Calories | 121.90 | 75.38 | 9.40 |
| Nutrients Calories as % of total | 58.7% | 36.3% | 4.5% |

In the context of the present disclosure, “the meal” refers to the total calorie intake during the period specified in the present disclosure, such as the total calorie intake from about 0 to 8 hours prior to or up to about 2 hours subsequent to administration of the MRI composition.

In some embodiments, the meal constitutes a plurality of food items, such as a toast with butter and a juice. In some embodiments, the subject has consumed no more than about 500 kcal from about 0 to 8 hours prior to or up to about 2 hours subsequent to administration of the MRI composition. In some embodiments, the subject has consumed no more than about 400 kcal from about 0 to 8 hours prior to or up to about 2 hours subsequent to administration of the MRI composition. In some embodiments, the subject has consumed no more than about 300 kcal from about 0 to 8 hours prior to or up to about 2 hours subsequent to administration of the MRI composition.

In some embodiments, the subject has consumed no more than about 500 kcal from the time specified herein prior to or up to the time specified herein subsequent to administration of the MRI composition.

In one embodiment, the meal comprises a predefined amount of carbohydrates, a predefined amount of fats, and a predefined amount of proteins.

In one embodiment, the predefined amount of carbohydrates in the meal is from 20 gram to 50 gram, such as from 20 to 22 gram, such as from 22 to 24 gram, such as from 24 to 26 gram, such as from 26 to 28 gram, such as from 28 to 30 gram, such as from 30 to 32 gram, such as from 32 to 34 gram, such as from 34 to 36 gram, such as from 36 to 38 gram, such as from 38 to 40 gram, such as from 40 to 42 gram, such as from 42 to 44 gram, such as from 44 to 46 gram, such as from 46 to 48 gram, such as from 48 to 50 gram.

In one embodiment, the predefined amount of carbohydrates in the meal is about 30 gram or about 31 gram.

In one embodiment, the predefined amount of carbohydrates in the meal is from 60 to 200 kcal, such as from 60 to 70 kcal, such as from 70 to 80 kcal, such as from 80 to 90 kcal, such as from 90 to 100 kcal, such as from 100 to 110 kcal, such as from 110 to 120 kcal, such as from 120 to 130 kcal, such as from 130 to 140 kcal, such as from 140 to 150 kcal, such as from 150 to 160 kcal, such as from 160 to 170 kcal, such as from 170 to 180 kcal, such as from 180 to 190 kcal, such as from 190 to 200 kcal.

In one embodiment, the predefined amount of carbohydrates in the meal is from 50 to 70 kcal % of the total amount of calories in the meal, such as from 50 to 52 kcal %, such as from 52 to 54 kcal %, such as from 54 to 56 kcal %, such as from 56 to 58 kcal %, such as from 58 to 60 kcal %, such as from 60 to 62 kcal %, such as from 62 to 64 kcal %, such as from 64 to 66 kcal %, such as from 66 to 68 kcal %, such as from 68 to 70 kcal % of the total amount of calories in the meal.

In one embodiment, the predefined amount of carbohydrates in the meal is about 59% of the total amount of calories in the meal.

In one embodiment, the predefined amount of fats in the meal is from 5 gram to 30 gram, such as from 5 to 10 gram, such as from 10 to 15 gram, such as from 15 to 20 gram, such as from 20 to 25 gram, such as from 25 to 30 gram.

In one embodiment, the predefined amount of fats in the meal is about 7 gram, about 8 gram, about 9 gram, or about 10 gram.

In one embodiment, the predefined amount of fats in the meal is from 30 to 150 kcal, such as from 30 to 40 kcal, such as from 40 to 50 kcal, such as from 50 to 60 kcal, such as from 60 to 70 kcal, such as from 70 to 80 kcal, such as from 80 to 90 kcal, such as from 90 to 100 kcal, such as from 100 to 110 kcal, such as from 110 to 120 kcal, such as from 120 to 130 kcal, such as from 130 to 140 kcal, such as from 140 to 150 kcal.

In one embodiment, the predefined amount of fats in the meal is from 25 to 45 kcal % of the total amount of calories in the meal, such as from 25 to 27 kcal %, such as from 27 to 29 kcal %, such as from 29 to 31 kcal %, such as from 31 to 33 kcal %, such as from 33 to 35 kcal %, such as from 35 to 37 kcal %, such as from 37 to 39 kcal %, such as from 39 to 41 kcal %, such as from 41 to 43 kcal %, such as from 43 to 45 kcal % of the total amount of calories in the meal.

In one embodiment, the predefined amount of fats in the meal is about 36% of the total amount of calories in the meal.

In one embodiment, the predefined amount of proteins in the meal is from 0 gram to 10 gram, such as from 0 to 2 gram, such as from 2 to 4 gram, such as from 4 to 6 gram, such as from 6 to 8 gram, such as from 8 to 10 gram.

In one embodiment, the predefined amount of proteins in the meal is about 0 gram, about 1 gram, about 2 gram, or about 3 gram.

In one embodiment, the predefined amount of proteins in the meal is from 0 to 50 kcal, such as from 0 to 5 kcal, such as from 5 to 10 kcal, such as from 10 to 15 kcal, such as from 15 to 20 kcal, such as from 20 to 25 kcal, such as from 25 to 30 kcal, such as from 30 to 35 kcal, such as from 35 to 40 kcal, such as from 40 to 45 kcal, such as from 45 to 50 kcal.

In one embodiment, the predefined amount of proteins in the meal is from 0 to 10 kcal % of the total amount of calories in the meal, such as from 0 to 1 kcal %, such as from 1 to 2 kcal %, such as from 2 to 3 kcal %, such as from 3 to 4 kcal %, such as from 4 to 5 kcal %, such as from 5 to 6 kcal %, such as from 6 to 7 kcal %, such as from 7 to 8 kcal %, such as from 8 to 9 kcal %, such as from 9 to 10 kcal % of the total amount of calories in the meal.

In one embodiment, the predefined amount of proteins in the meal is about 5% of the total amount of calories in the meal.

In one embodiment, the meal comprises one or more food items selected from the group consisting of: bread, such as toast bread with butter, and fruit juice, such as sweet lime juice.

In one embodiment, the meal comprises one or more sugars selected from the list consisting of: glucitol, mannitol, glycerol, glycol, α-glucose, α-galactose, α-xylose, β-glucose, β-galactose, β-xylose, β-arabinose and sucrose.

In one embodiment, the juice is provided at a volume of from 100 mL to 400 mL, such as from 100 mL to 110 mL, such as from 110 mL to 120 mL, such as from 120 mL to 130 mL, such as from 130 mL to 140 mL, such as from 140 mL to 150 mL, such as from 150 mL to 160 mL, such as from 160 mL to 170 mL, such as from 170 mL to 180 mL, such as from 180 mL to 190 mL, such as from 190 mL to 200 mL, such as from 200 mL to 210 mL, such as from 210 mL to 220 mL, such as from 220 mL to 230 mL, such as from 230 mL to 240 mL, such as from 240 mL to 250 mL, such as from 250 mL to 260 mL, such as from 260 mL to 270 mL, such as from 270 mL to 280 mL, such as from 280 mL to 290 mL, such as from 290 mL to 300 mL, such as from 300 mL to 310 mL, such as from 310 mL to 320 mL, such as from 320 mL to 330 mL, such as from 330 mL to 340 mL, such as from 340 mL to 350 mL, such as from 350 mL to 360 mL, such as from 360 mL to 370 mL, such as from 370 mL to 380 mL, such as from 380 mL to 390 mL, such as from 390 mL to 400 mL.

In one embodiment, the calorie content of the meal is from 100 kcal to 500 kcal, such as from 100 to 110 kcal, such as from 110 kcal to 120 kcal, such as from 120 kcal to 130 kcal, such as from 130 kcal to 140 kcal, such as from 140 kcal to 150 kcal, such as from 150 kcal to 160 kcal, such as from 160 kcal to 170 kcal, such as from 170 kcal to 180 kcal, such as from 180 kcal to 190 kcal, such as from 190 kcal to 200 kcal, such as from 200 kcal to 210 kcal, such as from 210 kcal to 220 kcal, such as from 220 kcal to 230 kcal, such as from 230 kcal to 240 kcal, such as from 240 kcal to 250 kcal, such as from 250 kcal to 260 kcal, such as from 260 kcal to 270 kcal, such as 270 kcal to 280 kcal, such as from 280 kcal to 290 kcal, such as from 290 kcal to 300 kcal, such as from 300 kcal to 310 kcal, such as from 310 kcal to 320 kcal, such as from 320 kcal to 330 kcal, such as from 330 kcal to 340 kcal, such as from 340 kcal to 350 kcal, such as from 350 kcal to 360 kcal, such as from 360 kcal to 370 kcal, such as from 370 kcal to 380 kcal, such as from 380 kcal to 390 kcal, such as from 390 kcal to 400 kcal, such as from 400 kcal to 410 kcal, such as from 410 kcal to 420 kcal, such as from 420 kcal to 430 kcal, such as from 430 kcal to 440 kcal, such as from 440 kcal to 450 kcal, such as from 450 kcal to 460 kcal, such as from 460 kcal to 470 kcal, such as from 470 kcal to 480 kcal, such as from 480 kcal to 490 kcal, such as from 490 kcal to 500 kcal.

In a particular embodiment, the meal has a calorie content of about 300 kcal or below, and the predefined amount of carbohydrates in the meal constitutes at least 50% of the calorie content of the meal.

In some embodiments, the meal employed has a calorie content of about 300 kcal or below, and the predefined amount of carbohydrates in the meal constitutes at least 50% of the calorie content of the meal, and the subject has consumed the meal from 0 to 4 hours prior to administration of the MRI composition, such as from 0 to 1 hours, such as from 1 to 2 hours, such as from 2 to 3 hours, such as from 3 to 4 hours prior to the administration of the MRI composition.

In some embodiments, the meal does not comprise certain food items, metal ions, or metals as specified herein below.

In some embodiments, the meal does not comprise xanthine or derivatives thereof, such as caffeine. In some embodiments, the meal does not comprise one or more food items selected from the group consisting of: chocolate, tea, coffee, cola, and tobacco. In some embodiments, the meal does not comprise dietary supplementation, such as vitamin supplementation.

In some embodiments, the meal does not comprise metal ions, such as metal irons derived from one or more of: calcium, chromium, copper, iron, magnesium, manganese, molybdenum, potassium, sodium and zinc.

In some embodiments, the meal does not comprise iron in an amount of 10 mg or more, such as 15 mg or more.

In some embodiments, the meal does not comprise magnesium in an amount of 100 mg or more, such as 200 mg or more, such as 300 mg or more, such as 400 mg or more.

In some embodiments, the meal does not comprise calcium in an amount of 500 mcg or more, such as 1000 mcg or more.

In some embodiments, the meal does not comprise calcium, magnesium and/or iron in the amounts specified herein.

In some embodiments, the meal does not comprise one or more of magnesium-containing antacids and one or more laxatives. In some embodiments, the meal does not comprise grapefruit or food products derived from grapefruit. In some embodiments, the meal does not comprise ethanol.

In some embodiments, the meal does not comprise one or more of the food items, metal ions, or metals in any combination or amounts specified herein.

### Methods and Effects

In one embodiment, a method for MRI of a subject is provided comprising, administering a composition comprising separately or together, a physiologically acceptable manganese (II) compound, a proteinogenic amino acid and/or a vitamin D, and optionally one or more water-soluble excipients to the subject; and performing MRI on said subject; wherein the subject has consumed a meal from 0 to 8 hours prior to or up to 2 hours subsequent to administration of the MRI composition, and wherein the calorie content of the meal is about 500 kcal or below.

The MRI is usually performed after a defined period of time following administration of the MRI composition to the subject as described elsewhere herein.

In one embodiment, a method of reducing or preventing nausea, diarrhea, and/or headaches in a subject receiving an MRI composition is provided comprising, separately or together, a physiologically acceptable manganese (II) compound, a proteinogenic amino acid and/or a vitamin D, and optionally one or more water-soluble excipients; the method comprising:
i) administering a meal to the subject; and
ii) administering the MRI composition to the subject;
wherein the meal is administered from 0 to 8 hours prior to or up to 2 hours subsequent to administering the MRI composition.

In one embodiment, the meal is administered from 0 to 7 hours prior to the administering of the MRI composition, such as from 0 to 1 hour, such as from 1 to 2 hours, such as from 2 to 3 hours, such as from 3 to 4 hours, such as from 4 to 5 hours, such as from 5 to 6 hours, such as from 6 to 7 hours prior to the administering of the MRI composition.

In one embodiment, the imaging quality is unaffected compared to a method where the MRI composition is administered to a fasting subject, wherein the fasting subject has not been eating for more than 8 hours prior to administration of the MRI composition nor before MRI. Imaging quality is in some embodiments assessed in terms of signal intensity SI (%) enhancement.

### MRI Composition

In one embodiment, the MRI composition comprises, separately or together, a physiologically acceptable manganese (II) compound, a proteinogenic amino acid and/or a vitamin D, and optionally one or more water-soluble excipients.

### Physiologically Acceptable Manganese (II) Compounds

In one embodiment, the MRI composition is provided as defined herein, wherein the physiologically acceptable manganese (II) compound is a salt of an inorganic anion or an organic anion. In one embodiment, the inorganic anion is selected from the group consisting of chloride, fluoride, bromide, iodide, sulphate, and phosphate. In one embodiment, the organic anion is selected from the group consisting of: ascorbate, kojate, salicylate, and gluconate.

In one embodiment, the MRI composition is provided as defined herein, wherein the physiologically acceptable manganese (II) compound is selected from the group consisting of: manganese (II) sulphate, manganese (II) gluconate, manganese (II) chloride anhydrate, manganese (II) chloride dihydrate, manganese (II) chloride tetrahydrate, and a combination thereof.

In one embodiment, the MRI composition is provided as defined herein, wherein the physiologically acceptable manganese (II) compound is manganese (II) chloride tetrahydrate or another hydrate of manganese (II) chloride.

In one embodiment, the MRI composition is provided as defined herein, wherein the MRI composition comprises between 0.5 g and 1.2 g manganese (II) chloride tetrahydrate or an equimolar amount of any corresponding manganese (II) salt.

In one embodiment, the MRI composition is provided as defined herein, wherein the MRI composition comprises between 0.6 g and 1 g of manganese (II) chloride tetrahydrate, such as between 0.65 g and 0.95 g, such as between 0.70 g and 0.90 g, such as between 0.75 g and 0.85 g, such as 0.80 g.

In one embodiment, the MRI composition is provided as defined herein, wherein the MRI composition comprises 0.8 g manganese (II) chloride tetrahydrate or an equimolar amount of any corresponding manganese (II) salt.

In one embodiment, the MRI composition is provided as defined herein, wherein the MRI composition comprises 0.8 g manganese (II) chloride tetrahydrate or an equimolar amount of the corresponding anhydrate or dihydrate.

In some embodiments, the MRI composition is Orviglance comprising: 800 mg manganese (II) chloride tetrahydrate, 500 mg L-alanine, and 800 IU vitamin D3. Orviglance has previously been known as Mangoral. In some embodiments, the MRI composition further comprises one or more water-soluble excipients.

In some embodiments, the MRI composition comprises: manganese (II) chloride tetrahydrate, L-alanine, and vitamin D3. In some embodiments, the MRI composition further comprises one or more water-soluble excipients.

In some embodiments, the MRI composition comprises: manganese (II) chloride or any hydrate thereof, L-alanine, and vitamin D3. In some embodiments, the MRI composition further comprises one or more water-soluble excipients.

### Pharmaceutical Excipients

A pharmaceutical excipient is a substance formulated alongside the active ingredient of a medicament, included for the purpose of long-term stabilization, bulking up solid formulations that contain potent active ingredients in small amounts (thus often referred to as “bulking agents”, “fillers”, or “diluents”), or to confer a therapeutic enhancement on the active ingredient in the final dosage form, such as but not limited to facilitating drug absorption, reducing viscosity, or enhancing solubility. Pharmaceutical excipients can also be useful in the manufacturing process, to aid in the handling of the active substance concerned such as by facilitating powder flowability or non-stick properties, in addition to aiding in vitro stability such as prevention of denaturation or aggregation over the expected shelf life.

It will be understood by a person of skill in the art, that while some excipients are preferred and some are not preferred to include in the compositions as disclosed herein, it may be possible to produce an MRI composition as defined herein (ie provided together or separate) non-preferred excipient if the relative amount of that excipient is carefully controlled.

In one embodiment, the MRI composition is provided as defined herein, wherein the one or more water-soluble excipients are non-hygroscopic water-soluble excipients. In one embodiment, the one or more water-soluble excipients are selected from the group consisting of: a non-hygroscopic filler, a non-hygroscopic binder, a non-hygroscopic disintegrant, and a non-hygroscopic lubricant.

Fillers/diluents: a “filler” or a “diluent” according to the present disclosure is preferably a water-soluble and non-hygroscopic filler, but may in some embodiments also be a hygroscopic filler. In one embodiment, the MRI composition is provided as defined herein, wherein the non-hygroscopic filler is selected from the group consisting of: isomalt; lactose, such as spray-dried lactose, α-lactose, or β-lactose; maltitol; maltose; and mannitol.

Dextrin and lactitol may be used as alternative fillers in low amounts but typically contain 5% moisture.

Dextrates, dextrose, fructose, hypromellose, maltodextrin, sucrose, sorbitol and xylitol are also water-soluble fillers but are all hygroscopic. These may thus be used in some embodiments where hygroscobicity is not a relevant issue, such as when the MRI composition is formulated in separate compartments. In some embodiments, the MRI composition is provided as defined herein, wherein the MRI composition comprise a hygroscopic filler selected from the group consisting of: a dextrate, dextrose, fructose, hypromellose, maltodextrin, sucrose, sorbitol and xylitol.

In some embodiments, the MRI composition may comprise a water-insoluble filler such as selected from the group consisting of: calcium carbonate, calcium phosphate (e.g. basic calcium phosphate, calcium hydrogen phosphate, dicalcium phosphate), calcium silicate, cellulose powdered, cellulose acetate, magnesium carbonate, magnesium oxide, medium-chain triglycerides, microcrystalline cellulose, silicified microcrystalline cellulose, polymethacrylates, starch, calcium sulfate, and pregelatinized starch. In other embodiments, the MRI composition is provided as defined herein, wherein the MRI composition does not comprise a water-insoluble filler selected from the group consisting of calcium carbonate; calcium phosphate, such as basic calcium phosphate, calcium hydrogen phosphate, or dicalcium phosphate; calcium silicate; cellulose powder; cellulose acetate; magnesium carbonate; magnesium oxide; a medium-chain triglyceride; microcrystalline cellulose; silicified microcrystalline cellulose; a polymethacrylate; starch; calcium sulfate; and pregelatinized starch.

Binders: a “binder” according to the present disclosure is preferably a water-soluble and non-hygroscopic binder. In one embodiment, the water-soluble and non-hygroscopic binder is selected from the group consisting of: hydroxyethylmethyl cellulose, maltose, povidone, and dextrin. In one embodiment, the binder is povidone. Binders are in particular of relevance when the contents of the MRI composition as defined herein in some embodiments are formulated together such as in a tablet.

Carboxymethylcellulose sodium, dextrates, dextrose, hydroxyethyl cellulose, hydroxypropyl cellulose, maltodextrin, poloxamer, polydextrose and sucrose may be used as alternative water-soluble binders in low amounts but are all hygroscopic.

In one embodiment, the MRI composition is provided as defined herein, wherein the MRI composition does not comprise a poorly water-soluble binder selected from the group consisting of: acacia, hypromellose, methylcellulose, sodium alginate, pre-gelatinized starch, inulin, agar, gelatin, starch, alginic acid, cellulose acetate phthalate, ethylcellulose, hydrogenated vegetable oil, magnesium aluminum silicate, microcrystalline cellulose, and polymethacrylate.

Disintegrants: a “disintegrant” is known in the art to expand and dissolve when wet causing a compressed pharmaceutical composition, such as a tablet, to break apart.

Disintegrants are thus in particular of relevance when the MRI composition as defined herein is formulated together such as in a tablet.

Povidone is a water-soluble disintegrant, but not a very strong disintegrant. In some embodiments, the MRI composition is provided comprising a non-hygroscopic disintegrant, wherein the non-hygroscopic disintegrant is povidone.

Carboxymethylcellulose calcium, croscarmellose sodium, and crospovidone are insoluble in water, but very strong disintegrants and may be applied in very low levels and are preferred. In some embodiments, the MRI composition is provided as defined herein, further comprising a water-insoluble disintegrant, such as selected from the group consisting of: carboxymethylcellulose calcium; croscarmellose sodium; and crospovidone.

Carboxymethylcellulose sodium and hydroxypropyl cellulose are examples of hygroscopic water-soluble disintegrants. These may thus be used in some embodiments where hygroscobicity is not a relevant issue, such as when the MRI composition is formulated in separate compartments. In one embodiment, the MRI composition comprise a hygroscopic disintegrant selected from the group consisting of: carboxymethylcellulose sodium and hydroxypropyl cellulose.

Lubricants: A “lubricant” prevents ingredients from sticking to and clogging production machinery, such as filling tubes and tablet punches. E.g. during tablet production, lubricants also ensure that tablet formation and ejection can occur with low friction between the solid and die wall.

Polyethylene glycol 6000 and sodium benzoate are water-soluble and non-hygroscopic binders. In one embodiment, the MRI composition is provided as defined herein, wherein the non-hygroscopic lubricant is selected from the group consisting of: polyethylene glycol 6000 and sodium benzoate.

Sodium lauryl sulfate yields a bitter taste and hence, is not preferred.

Poloxamer and polyethylene glycol 4000 are water-soluble but hygroscopic lubricants and may thus be used in some embodiments where hygroscobicity is not a relevant issue, such as when the MRI composition is formulated in separate compartments. In some embodiments, the MRI composition is provided as defined herein, wherein the MRI composition comprise a hygroscopic lubricant selected from the group consisting of: poloxamer and polyethylene glycol 4000.

Sodium stearyl fumarate, calcium stearate, colloidal silica, glycerin monostearate, glyceryl behenate, glyceryl palmitostearate, hydrogenated castor oil, hydrogenated vegetable oil, magnesium stearate, medium-chain triglycerides, palmitic acid, stearic acid, talc, zinc stearate are all water-insoluble and thus not preferred to include in the MRI composition as defined herein. In one embodiment, the MRI composition does not comprise a water-insoluble lubricant selected from the group consisting of: sodium stearyl fumarate, calcium stearate, colloidal silica, glycerin monostearate, glyceryl behenate, glyceryl palmitostearate, hydrogenated castor oil, hydrogenated vegetable oil, magnesium stearate, medium-chain triglycerides, palmitic acid, stearic acid, talc, and zinc stearate.

### Absorption Promoters

Under physiological circumstances the manganese of the physiologically acceptable manganese (II) compound is poorly absorbed from the intestine after oral intake, but by the use of specific absorption promoters, such as L-alanine and vitamin D3, it is possible to obtain a sufficiently high concentration in the liver in order to achieve a significant signal enhancing effect during MRI.

The term “absorption promoter” is used herein interchangeably with the term “uptake promoter” which refers to a compound capable of enhancing manganese transport across the membranes of the gastrointestinal tract. These compounds are well known in the art from e.g. WO 96/05867 and comprise physiologically tolerable reducing compounds containing an α-hydroxy ketone group, a physiologically tolerable acid containing α- and/or β-hydroxy or amino groups, or a salt thereof, and/or vitamin D.

In one embodiment, the MRI composition is provided as defined herein, wherein the one or more absorption promotors are selected from the group consisting of: a proteinogenic amino acid and a vitamin D.

In one embodiment, the MRI composition is provided as defined herein, wherein the proteinogenic amino acid is selected from the group consisting of alanine, valine, leucine, tryptophan, methionine, isoleucine, proline, phenylalanine, serine, glycine, threonine, cysteine, asparagine, glutamine, tyrosine, aspartic acid, glutamic acid, arginine, lysine and histidine.

In one embodiment, the MRI composition is provided as defined herein, wherein the proteinogenic amino acid is a hydrophobic amino acid selected from the group consisting of alanine, glcine, valine, leucine, isoleucine, proline, phenylalanine, methionine and tryptophan.

In one embodiment, the MRI composition is provided as defined herein, wherein the proteinogenic amino acid is a neutral amino acid.

In one embodiment, the MRI composition is provided as defined herein, wherein the proteinogenic amino acid is L-alanine.

In one embodiment, the MRI composition is provided as defined herein, wherein the vitamin D is vitamin D3.

In one embodiment, the MRI composition is provided as defined herein, comprising two absorptions promoters. In one embodiment, the two absorption promoters are L-alanine and vitamin D3.

In one embodiment, the MRI composition is provided as defined herein, wherein the MRI composition comprises between 0.25 g and 0.75 g of a proteinogenic amino acid, such as L-alanine, such as between 0.30 g and 0.70 g, such as between 0.35 g and 0.65 g, such as between 0.40 g and 0.60 g, such as between 0.45 g and 0.55 g, such as 0.50 g.

### Items

1. An MRI composition comprising, separately or together, a physiologically acceptable manganese (II) compound, a proteinogenic amino acid and/or a vitamin D, and one or more water-soluble excipients; wherein the MRI composition is for use in the diagnosis in vivo of liver metastases and/or primary liver cancer in a subject; wherein the subject has consumed a meal from 0 to 8 hours prior to or up to 2 hours subsequent to administration of the MRI composition and wherein the calorie content of the meal is about 500 kcal or below.

2. The MRI composition for use according to item 1, wherein the use reduces or prevents nausea, diarrhea, and/or headaches in the subject as compared to a fasting subject that has not consumed food for 3 hours or more prior to administration of the MRI composition.

3. The MRI composition for use according to any one of the preceding items, wherein the subject has not been fasting for 3 or more hours prior to the administration of the MRI composition.

4. The MRI composition for use according to any one of the preceding items, wherein the subject is negatively affected by not eating for more than 8 hours.

5. The MRI composition for use according to any one of the preceding items, wherein the subject suffers from a disease where not eating for more than 8 hours imposes a health risk to the subject.

6. The MRI composition for use according to any one of the preceding items wherein the subject has one or more of:
a. diabetes, such as diabetes type-I or diabetes type-II;
b. a Body Mass Index (BMI) of 20 or below;
c. an age of 60 years or above; and/or

7. The MRI composition for use according to any one of the preceding items, wherein the subject has consumed the meal from 0 to 7 hours prior to administration of the MRI composition, such as from 0 to 1 hour, such as from 1 to 2 hours, such as from 2 to 3 hours, such as from 3 to 4 hours, such as from 4 to 5 hours, such as from 5 to 6 hours, such as from 6 to 7 hours prior to administration of the MRI composition.

8. The MRI composition for use according to any one of the preceding items, wherein the subject has consumed the meal within about 1 hour prior to administration of the MRI composition, such as within about 45 minutes, such as within about 30 minutes prior to administration of the MRI composition.

9. The MRI composition for use according to any one of the preceding items, wherein the meal comprises a predefined amount of carbohydrates, a predefined amount of fats, and a predefined amount of proteins.

10. The MRI composition for use according to any one of the preceding items, wherein the meal is a snack.

11. The MRI composition for use according to any one of the preceding items, wherein the predefined amount of carbohydrates in the meal is from 20 gram to 50 gram, such as from 20 to 22 gram, such as from 22 to 24 gram, such as from 24 to 26 gram, such as from 26 to 28 gram, such as from 28 to 30 gram, such as from 30 to 32 gram, such as from 32 to 34 gram, such as from 34 to 36 gram, such as from 36 to 38 gram, such as from 38 to 40 gram, such as from 40 to 42 gram, such as from 42 to 44 gram, such as from 44 to 46 gram, such as from 46 to 48 gram, such as from 48 to 50 gram.

12. The MRI composition for use according to any one of the preceding items, wherein the predefined amount of carbohydrates in the meal is about 30 gram or about 31 gram.

13. The MRI composition for use according to any one of the preceding items, wherein the predefined amount of carbohydrates in the meal is from 60 to 200 kcal, such as from 60 to 70 kcal, such as from 70 to 80 kcal, such as from 80 to 90 kcal, such as from 90 to 100 kcal, such as from 100 to 110 kcal, such as from 110 to 120 kcal, such as from 120 to 130 kcal, such as from 130 to 140 kcal, such as from 140 to 150 kcal, such as from 150 to 160 kcal, such as from 160 to 170 kcal, such as from 170 to 180 kcal, such as from 180 to 190 kcal, such as from 190 to 200 kcal.

14. The MRI composition for use according to any one of the preceding items, wherein the predefined amount of carbohydrates in the meal is from 50 to 70 kcal % of the total amount of calories in the meal, such as from 50 to 52 kcal %, such as from 52 to 54 kcal %, such as from 54 to 56 kcal %, such as from 56 to 58 kcal %, such as from 58 to 60 kcal %, such as from 60 to 62 kcal %, such as from 62 to 64 kcal %, such as from 64 to 66 kcal %, such as from 66 to 68 kcal %, such as from 68 to 70 kcal % of the total amount of calories in the meal.

15. The MRI composition for use according to any one of the preceding items, wherein the predefined amount of carbohydrates in the meal is about 59% of the total amount of calories in the meal.

16. The MRI composition for use according to any one of the preceding items, wherein the predefined amount of fats in the meal is from 5 gram to 30 gram, such as from 5 to 10 gram, such as from 10 to 15 gram, such as from 15 to 20 gram, such as from 20 to 25 gram, such as from 25 to 30 gram.

17. The MRI composition for use according to any one of the preceding items, wherein the predefined amount of fats in the meal is about 7 gram, about 8 gram, about 9 gram, or about 10 gram.

18. The MRI composition for use according to any one of the preceding items, wherein the predefined amount of fats in the meal is from 30 to 150 kcal, such as from 30 to 40 kcal, such as from 40 to 50 kcal, such as from 50 to 60 kcal, such as from 60 to 70 kcal, such as from 70 to 80 kcal, such as from 80 to 90 kcal, such as from 90 to 100 kcal, such as from 100 to 110 kcal, such as from 110 to 120 kcal, such as from 120 to 130 kcal, such as from 130 to 140 kcal, such as from 140 to 150 kcal.

19. The MRI composition for use according to any one of the preceding items, wherein the predefined amount of fats in the meal is from 25 to 45 kcal % of the total amount of calories in the meal, such as from 25 to 27 kcal %, such as from 27 to 29 kcal %, such as from 29 to 31 kcal %, such as from 31 to 33 kcal %, such as from 33 to 35 kcal %, such as from 35 to 37 kcal %, such as from 37 to 39 kcal %, such as from 39 to 41 kcal %, such as from 41 to 43 kcal %, such as from 43 to 45 kcal % of the total amount of calories in the meal.

20. The MRI composition for use according to any one of the preceding items, wherein the predefined amount of fats in the meal is about 36% of the total amount of calories in the meal.

21. The MRI composition for use according to any one of the preceding items, wherein the predefined amount of proteins in the meal is from 0 gram to 10 gram, such as from 0 to 2 gram, such as from 2 to 4 gram, such as from 4 to 6 gram, such as from 6 to 8 gram, such as from 8 to 10 gram.

22. The MRI composition for use according to any one of the preceding items, wherein the predefined amount of proteins in the meal is about 0 gram, about 1 gram, about 2 gram, or about 3 gram.

23. The MRI composition for use according to any one of the preceding items, wherein the predefined amount of proteins in the meal is from 0 to 50 kcal, such as from 0 to 5 kcal, such as from 5 to 10 kcal, such as from 10 to 15 kcal, such as from 15 to 20 kcal, such as from 20 to 25 kcal, such as from 25 to 30 kcal, such as from 30 to 35 kcal, such as from 35 to 40 kcal, such as from 40 to 45 kcal, such as from 45 to 50 kcal.

24. The MRI composition for use according to any one of the preceding items, wherein the predefined amount of proteins in the meal is from 0 to 10 kcal % of the total amount of calories in the meal, such as from 0 to 1 kcal %, such as from 1 to 2 kcal %, such as from 2 to 3 kcal %, such as from 3 to 4 kcal %, such as from 4 to 5 kcal %, such as from 5 to 6 kcal %, such as from 6 to 7 kcal %, such as from 7 to 8 kcal %, such as from 8 to 9 kcal %, such as from 9 to 10 kcal % of the total amount of calories in the meal.

25. The MRI composition for use according to any one of the preceding items, wherein the predefined amount of proteins in the meal is about 5% of the total amount of calories in the meal.

26. The MRI composition for use according to any one of the preceding items, wherein the meal comprises one or more food items selected from the group consisting of: bread, such as toast bread, and fruit juice, such as lime juice.

27. The MRI composition for use according to any one of the preceding items, wherein the juice is provided at a volume of from 100 mL to 400 mL, such as from 100 mL to 110 mL, such as from 110 mL to 120 mL, such as from 120 mL to 130 mL, such as from 130 mL to 140 mL, such as from 140 mL to 150 mL, such as from 150 mL to 160 mL, such as from 160 mL to 170 mL, such as from 170 mL to 180 mL, such as from 180 mL to 190 mL, such as from 190 mL to 200 mL, such as from 200 mL to 210 mL, such as from 210 mL to 220 mL, such as from 220 mL to 230 mL, such as from 230 mL to 240 mL, such as from 240 mL to 250 mL, such as from 250 ml to 260 mL, such as from 260 mL to 270 mL, such as from 270 mL to 280 mL, such as from 280 mL to 290 mL, such as from 290 mL to 300 mL, such as from 300 mL to 310 mL, such as from 310 mL to 320 mL, such as from 320 mL to 330 mL, such as from 330 mL to 340 mL, such as from 340 mL to 350 mL, such as from 350 mL to 360 mL, such as from 360 mL to 370 mL, such as from 370 mL to 380 mL, such as from 380 mL to 390 mL, such as from 390 mL to 400 mL.

28. The MRI composition for use according to any one of the preceding items, wherein the calorie content of the meal is from 100 kcal to 500 kcal, such as from 100 to 110 kcal, such as from 110 kcal to 120 kcal, such as from 120 kcal to 130 kcal, such as from 130 kcal to 140 kcal, such as from 140 kcal to 150 kcal, such as from 150 kcal to 160 kcal, such as from 160 kcal to 170 kcal, such as from 170 kcal to 180 kcal, such as from 180 kcal to 190 kcal, such as from 190 kcal to 200 kcal, such as from 200 kcal to 210 kcal, such as from 210 kcal to 220 kcal, such as from 220 kcal to 230 kcal, such as from 230 kcal to 240 kcal, such as from 240 kcal to 250 kcal, such as from 250 kcal to 260 kcal, such as from 260 kcal to 270 kcal, such as 270 kcal to 280 kcal, such as from 280 kcal to 290 kcal, such as from 290 kcal to 300 kcal, such as from 300 kcal to 310 kcal, such as from 310 kcal to 320 kcal, such as from 320 kcal to 330 kcal, such as from 330 kcal to 340 kcal, such as from 340 kcal to 350 kcal, such as from 350 kcal to 360 kcal, such as from 360 kcal to 370 kcal, such as from 370 kcal to 380 kcal, such as from 380 kcal to 390 kcal, such as from 390 kcal to 400 kcal.

29. The MRI composition for use according to any one of the preceding items, wherein the calorie content of the meal is about 300 kcal or below, and wherein the predefined amount of carbohydrates in the meal constitutes at least 50% of the calorie content of the meal.

30. A method for Magnetic Resonance Imaging (MRI) of a subject comprising, administering a composition comprising separately or together, a physiologically acceptable manganese (II) compound, a proteinogenic amino acid and/or a vitamin D, and one or more water-soluble excipients to the subject; and performing MRI on said subject; wherein the subject has consumed a meal from 0 to 8 hours prior to or up to 2 hours subsequent to administration of the MRI composition, and wherein the calorie content of the meal is about 500 kcal or below.

31. A method of reducing or preventing nausea, diarrhea, and/or headaches in a subject receiving an MRI composition comprising, separately or together, a physiologically acceptable manganese (II) compound, a proteinogenic amino acid and/or a vitamin D, and one or more water-soluble excipients; the method comprising:
i) administering a meal to the subject; and
ii) administering the MRI composition to the subject;
wherein the meal is administered from 0 to 8 hours prior to or up to 2 hours subsequent to administering the MRI composition.

32. The method according to item 31, wherein the meal is administered from 0 to 6 hours prior to the administering of the MRI composition, such as from 0 to 1 hour, such as from 1 to 2 hours, such as from 2 to 3 hours, such as from 3 to 4 hours, such as from 4 to 5 hours, such as from 5 to 6 hours prior to the administering of the MRI composition.

33. The method according to any one of items 30 to 32, wherein the imaging quality is unaffected compared to a method where the MRI composition is administered to a fasting subject, wherein the fasting subject has not been eating for more than 8 hours.

### Examples

### Example 1: Study of Orviglance Food Effects

### Materials

### Active Treatment

IMP: Manganese (II) chloride tetrahydrate (MnCl2·4H2O; working name: Orviglance) powder for oral administration. Single oral dose of Orviglance (800 mg manganese (II) chloride tetrahydrate, 500 mg L-alanine, and 800 IU vitamin D3).

### General Pharmacology:

Orviglance is an oral liver imaging drug (i.e. a liver specific contrast agent) being developed for detection and visualization of focal liver lesions, using Magnetic Resonance Imaging (“MRI”) in patients where use of gadolinium-based contrast agents (“GBCAs”) may be medically inadvisable or cannot be administered.

### Overall Objective:

To evaluate effects of food on Orviglance after a single oral administration in adult healthy subjects. To assess the PK and PD of Orviglance in healthy human subjects in fasting and fed (full meal or snacks) conditions.

### Method

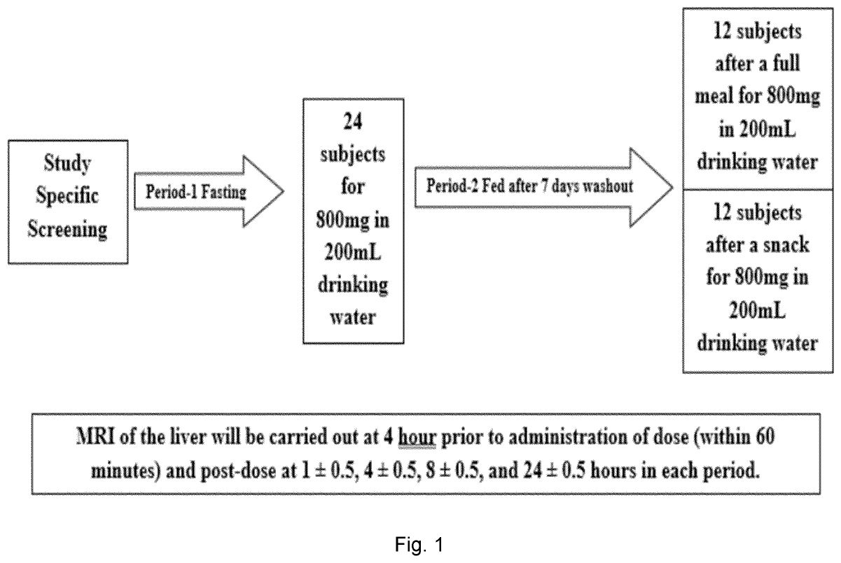

Period-1 was under fasting condition and Period-2 was under fed condition (full meal according to Table 1, or a snack according to Table 2). A flow chart of the study design is also depicted in FIG. 1. The rationale for the study design was to allow evaluation of orally administered Orviglance under fasting and fed conditions (full meal or snack), respectively by assessing Pharmacokinetics (PK) and Pharmacodynamics (PD) in healthy adult human subjects c.f. FIG. 1.

### Randomization

The allocation to receive light snack and full meal for each subject during second period of the study was determined according to a randomization schedule. The randomization schedule was generated using SAS® Version 9.4 or higher (SAS Institute Inc., USA) by the biostatistician. The sequence of administration of treatments i.e. “T1T21” or “T1T22” to the subjects was determined according to the randomization schedule as per below table 3. Equal allocation of subjects in each sequence was ensured.

TABLE 3
overview of randomization schedule for Example 1.

| Sample size | Period-1 | Period-2 |
|---|---|---|
| (N = 24) | (Fasting, N = 24) | (Fed, N = 24) |
| Sequence T₁T₂₁ | Test 800 mg/200 mL (T₁) | Test 800 mg/200 mL |
| (N = 12) | | after full meal (T₂₁) |
| Sequence T₁T₂₂ | | Test 800 mg/200 mL |
| (N = 12) | | after snack (T₂₂) |

### Number of Subjects

Maximum 32 subjects were enrolled in the study in order to obtain data of 24 completers who completed both fasting and fed (full meal and snack) study periods.

### Housing

Subjects were housed in the clinical facility at least 60 hours before administration of the dose and continued to remain in the clinical facility for at least 72 hours after the administration of investigational medicinal product in all the periods.

Check-in procedures/Check-in activities were carried out as per in house procedure at each period. The subjects stayed in the facility for six consecutive nights in each period.

MRI of the liver was carried out at 4 hour prior to administration of dose (within 60 minutes) and post-dose at 1±0.5, 4±0.5, 8±0.5, and 24±0.5 hours utilizing imaging parameters.

### Diet

All subjects were to abstain from xanthine containing food or beverages (like chocolates, tea, coffee or cola drinks), tobacco, products containing tobacco (like pan, pan masala, gutkha), for 24 hours prior to check-in till their stay in clinical facility. Dietary iron supplementation, including a multivitamin that contains iron, was prohibited from 6 hours prior to check-in of period-1 until after the last contrast-enhanced MRI of study. Supplemental magnesium and/or supplemental calcium may reduce manganese absorption and was prohibited from 6 hours prior to check-in of period-1 until after the last contrast-enhanced MRI of study.

Supplemental vitamin C or vitamin D3, which promote manganese absorption, were prohibited from 6 hours prior to check-in of period-1 until after the last contrast enhanced MRI of study. Magnesium-containing antacids and laxatives were prohibited from 6 hours prior to check-in of period-1 until after the last contrast-enhanced MRI of study. Quinolone and tetracycline antibiotics were prohibited from 6 hours prior to check-in of period-1 until after the last contrast-enhanced MRI of study.

All subjects were to abstain from grapefruit or its products within 72 hours prior to check-in of period-1 till last sample collection of study. All subjects were to abstain from alcohol or alcoholic products, recreational drugs within 48 hours prior to check-in of period-1 till last sample collection of study. Smoking for 6 months prior to check-in of period-1 till the completion of the study was prohibited and the subjects were to abstain from the same.

### Postural Restriction

Orviglance was administered to the subjects while in sitting posture. Subjects were in sitting posture or ambulatory posture for the first 4 hours post-dose in each period. In case of adverse event(s), position of the subject was changed appropriately.

Thereafter, the subjects were allowed to engage only in normal activities while avoiding any strenuous physical activity. Note: Subjects were in supine position at the time of MRI.

### Duration of Fasting & Distribution of Meals:

### For Period-1:

An overnight fast of at least 4 hours prior to dosing in period-1 and lunch will be provided after completion of 4 hr post dose MRI.

### For Period-2:

All subjects will be required to fast overnight for at least 6 hours prior to serving of full meal according to Table 1 or a snack according to Table 2, which they are required to consume completely within 30 minutes of serving the same. Lunch will be provided after 4±1 hours of dose administration in period-2 after. The dietician/trained study person weighed the leftover food and calculated the calories for any subjects that had not consumed the full meal or snacks completely. Standardized meals were served to the subjects at appropriate times during their stay in the clinical facility. The subjects received lunch at least 4±1 hours (after 4 hours MRI) (for period-1 and for period-2) after dosing and further meals were served at appropriate intervals from then on, until checkout.

Subjects were to refrain from drinking water from 1 hour before till 1 hour after each dosing in each period except for 200 ml of water administered during dosing. Prior to and thereafter, water could be consumed as required.

### Results

The results of the study are shown in below tables, Table 4 (comparing full meal vs fasting); and Table 5 (comparing the snack vs fasting). Optimal SI (%) enhancement is most often found about 4 hours post dosing; hence, comparison of SI (%) at 4 hours post dosing is important to examine the difference in SI (%) between nonfasting and fasting subjects.

TABLE 4
Relative change in MRI signal intensity (SI %) and signal intensity from liver
imaging at 1, 4, 8 and 24 hours post-dose for fasting subjects (T1) compared to
the same subjects after receiving a full meal (T21). The SI % obtained from
imaging of the subjects after a full meal is significantly lower than
after the fasting period.
Mean (SD) (T21 - full meal vs T1 - fasting)

| | | | Normalised signal | | Liver | |
|---|---|---|---|---|---|---|
| | SNR | | intensity | | SI(%)enhancement | |
| | Test | Test | Test | Test | Test | Test |
| Statistics | (T1) | (T21) | (T1) | (T21) | (T1) | (T21) |
| Baseline | 37.9 | 179.3 | — | — | — | — |
| | (16.47) | (403.05) | | | | |
| at 1 hrs. | 98.2 | 129.1 | 129.4 | 148.4 | 0.4 | −28.2 |
| | (217.87) | (194.17) | (490.21) | (378.99) | (24.31) | (13.64) |
| at 4 hrs. | 208.0 | 77.7 | 381.0 | 27.0 | 20.0 | −7.7 |
| | (518.88) | (88.12) | (1172.65) | (43.35) | (16.34) | (14.99) |
| at 8 hrs. | 63.9 | 119.5 | 65.5 | 20.4 | 15.4 | 1.4 |
| | (56.75) | (183.24) | (129.17) | (64.54) | (25.20) | (18.28) |
| at 24 hrs. | 118.2 | 195.9 | 239.8 | 132.1 | −5.0 | −10.1 |
| | (130.88) | (321.88) | (407.34) | (260.89) | (20.87) | (18.23) |

TABLE 5
Relative change in MRI signal intensity (SI %) and signal intensity from liver
imaging at 1, 4, 8 and 24 hours post-dose for fasting subjects (T1) compared to
the same subjects after receiving a snack (T22). The SI % obtained from imaging
of the subject after the snack is surprisingly at least as good as after fasting.
Mean (SD) (T22 - snack vs T1 - fasting)

| | | | Normalised signal | | Liver | |
|---|---|---|---|---|---|---|
| | SNR | | intensity | | SI(%)enhancement | |
| | Test | Test | Test | Test | Test | Test |
| Statistics | (T1) | (T22) | (T1) | (T22) | (T1) | (T22) |
| Baseline | 53.7 | 128.9 | — | — | — | — |
| | (47.763) | (208.00) | | | | |
| at 1 hrs. | 48.3 | 91.8 | 22.5 | 5.3 | −13.3 | −6.1 |
| | (32.044) | (95.15) | (86.28) | (42.98) | (20.92) | (14.28) |
| at 4 hrs. | 239.8 | 60.3 | 320.8 | 28.5 | 12.6 | 17.7 |
| | (659.59) | (37.04) | (983.32) | (95.92) | (19.02) | (20.09) |
| at 8 hrs. | 63.9 | 148.3 | 69.4 | 51.0 | 1.4 | 5.6 |
| | (30.63) | (202.45) | (88.68) | (70.41) | (15.13) | (25.88) |
| at 24 hrs. | 194.9 | 78.7 | 572.5 | 4.0 | −16.6 | −1.8 |
| | (472.93) | (95.04) | (1786.19) | (52.90) | (19.50) | (29.55) |

### CONCLUSIONS

The present study supports the existing hypothesis that consumption of a full meal according to Table 1 prior to dosing of an oral manganese based MRI agent, such as Orviglance, significantly retards the imaging quality from subsequent liver MRI.

Surprisingly, the present study also supports that subjects having consumed a meal with a calorie content of about 500 kcal or below (a snack) are still able to undergo MRI using Orviglance with an imaging quality being at least as high as for fasting subjects. These findings significantly support the diagnostic potential of Orviglance, in particular for subjects where fasting can be inconvenient or problematic.

### Items 2

1. An MRI composition comprising, separately or together, a physiologically acceptable manganese (II) compound, a proteinogenic amino acid and/or a vitamin D, and one or more water-soluble excipients; wherein the MRI composition is for use in the diagnosis in vivo of liver metastases and/or primary liver cancer in a subject; wherein the subject has consumed a meal from 0 to 8 hours prior to or up to about 2 hours subsequent to administration of the MRI composition and wherein the calorie content of the meal is about 500 kcal or below.

2. The MRI composition for use according to item 1, wherein the use reduces or prevents nausea, diarrhea, and/or headaches in the subject as compared to a fasting subject that has not consumed food for 3 hours or more prior to administration of the MRI composition.

3. The MRI composition for use according to any one of the preceding items, wherein the subject has not been fasting for 3 or more hours prior to the administration of the MRI composition.

4. The MRI composition for use according to any one of the preceding items, wherein the subject is negatively affected by not eating for more than 8 hours.

5. The MRI composition for use according to any one of the preceding items, wherein the subject suffers from a disease where not eating for more than 8 hours imposes a health risk to the subject.

6. The MRI composition for use according to any one of the preceding items wherein the subject has one or more of:
a. diabetes, such as diabetes type-I or diabetes type-II;
b. a Body Mass Index (BMI) of 20 or below;
c. an age of 60 years or above; and/or

7. The MRI composition for use according to any one of the preceding items, wherein the subject has consumed the meal from 0 to 7 hours prior to administration of the MRI composition, such as from 0 to 1 hour, such as from 1 to 2 hours, such as from 2 to 3 hours, such as from 3 to 4 hours, such as from 4 to 5 hours, such as from 5 to 6 hours, such as from 6 to 7 hours prior to administration of the MRI composition.

8. The MRI composition for use according to any one of the preceding items, wherein the meal comprises a predefined amount of carbohydrates, a predefined amount of fats, and a predefined amount of proteins.

9. The MRI composition for use according to item 8, wherein the predefined amount of carbohydrates in the meal is from 20 gram to 50 gram, such as from 20 to 22 gram, such as from 22 to 24 gram, such as from 24 to 26 gram, such as from 26 to 28 gram, such as from 28 to 30 gram, such as from 30 to 32 gram, such as from 32 to 34 gram, such as from 34 to 36 gram, such as from 36 to 38 gram, such as from 38 to 40 gram, such as from 40 to 42 gram, such as from 42 to 44 gram, such as from 44 to 46 gram, such as from 46 to 48 gram, such as from 48 to 50 gram.

10. The MRI composition for use according to any one of items 8 to 9, wherein the predefined amount of fats in the meal is from 5 gram to 30 gram, such as from 5 to 10 gram, such as from 10 to 15 gram, such as from 15 to 20 gram, such as from 20 to 25 gram, such as from 25 to 30 gram.

11. The MRI composition for use according to any one of items 8 to 10, wherein the predefined amount of proteins in the meal is from 0 gram to 10 gram, such as from 0 to 2 gram, such as from 2 to 4 gram, such as from 4 to 6 gram, such as from 6 to 8 gram, such as from 8 to 10 gram.

12. The MRI composition for use according to any one of the preceding items, wherein the meal comprises one or more food items, such as bread, such as toast bread, and/or fruit juice, such as lime juice.

13. The MRI composition for use according to any one of the preceding items, wherein the calorie content of the meal is from 100 kcal to 400 kcal, such as from 100 to 110 kcal, such as from 110 kcal to 120 kcal, such as from 120 kcal to 130 kcal, such as from 130 kcal to 140 kcal, such as from 140 kcal to 150 kcal, such as from 150 kcal to 160 kcal, such as from 160 kcal to 170 kcal, such as from 170 kcal to 180 kcal, such as from 180 kcal to 190 kcal, such as from 190 kcal to 200 kcal, such as from 200 kcal to 210 kcal, such as from 210 kcal to 220 kcal, such as from 220 kcal to 230 kcal, such as from 230 kcal to 240 kcal, such as from 240 kcal to 250 kcal, such as from 250 kcal to 260 kcal, such as from 260 kcal to 270 kcal, such as 270 kcal to 280 kcal, such as from 280 kcal to 290 kcal, such as from 290 kcal to 300 kcal, such as from 300 kcal to 310 kcal, such as from 310 kcal to 320 kcal, such as from 320 kcal to 330 kcal, such as from 330 kcal to 340 kcal, such as from 340 kcal to 350 kcal, such as from 350 kcal to 360 kcal, such as from 360 kcal to 370 kcal, such as from 370 kcal to 380 kcal, such as from 380 kcal to 390 kcal, such as from 390 kcal to 400 kcal.

14. The MRI composition for use according to any one of the preceding items, wherein the physiologically acceptable manganese (II) compound is selected from the group consisting of: manganese (II) sulphate, manganese (II) gluconate, manganese (II) chloride anhydrate, manganese (II) chloride dihydrate, manganese (II) chloride tetrahydrate, and a combination thereof, preferably wherein the physiologically acceptable manganese (II) compound is manganese (II) chloride tetrahydrate or another hydrate of manganese (II) chloride

15. The MRI composition for use according to any one of the preceding items, wherein the proteinogenic amino acid is a hydrophobic amino acid selected from the group consisting of alanine, glcine, valine, leucine, isoleucine, proline, phenylalanine, methionine and tryptophan.

16. The MRI composition for use according to any one of the preceding items, wherein the calorie content of the meal is about 300 kcal or below, and wherein the predefined amount of carbohydrates in the meal constitutes at least 50% of the calorie content of the meal.

17. A method of reducing or preventing nausea, diarrhea, and/or headaches in a subject receiving an MRI composition comprising, separately or together, a physiologically acceptable manganese (II) compound, a proteinogenic amino acid and/or a vitamin D, and one or more water-soluble excipients; the method comprising:
i) administering a meal to the subject; and
ii) administering the MRI composition to the subject;
wherein the meal is administered from 0 to 8 hours prior to or up to about 2 hours subsequent to administering the MRI composition.

1. An MRI composition comprising, separately or together, manganese (II) chloride tetrahydrate, L-alanine, and a vitamin D; wherein the MRI composition is for use in the diagnosis in vivo of liver metastases and/or primary liver cancer in a subject; wherein the subject has consumed a meal from 0 to 8 hours prior to or up to 2 hours subsequent to administration of the MRI composition and wherein the calorie content of the meal is about 500 kcal or below.

2. The MRI composition for use according to claim 1, wherein the composition further comprises one or more water-soluble excipients.

3. The MRI composition for use according to any one of the preceding claims, wherein the subject has reduced nausea, diarrhea, and/or headaches as compared to a fasting subject having been administered said MRI composition.

4. The MRI composition for use according to claim 3, wherein the subject has reduced nausea and/or diarrhea as compared to a fasting subject having been administered said MRI composition.

5. The MRI composition for use according to any one of the preceding claims, wherein the subject has not been fasting for 3 or more hours prior to the administration of the MRI composition.

6. The MRI composition for use according to any one of the preceding claims, wherein the subject is classified as a subject that is negatively affected by not eating for more than 8 hours.

7. The MRI composition for use according to any one of the preceding claims, wherein the subject suffers from a disease where not eating for more than 8 hours imposes a health risk to the subject.

8. The MRI composition for use according to any one of the preceding claims, wherein the subject has one or more of:
a. diabetes, such as diabetes type-I or diabetes type-II;
b. a Body Mass Index (BMI) of 20 or below;
c. an age of 60 years or above; and/or
d. hyperthyroidism.

9. The MRI composition for use according to any one of the preceding claims, wherein the subject has consumed the meal from 0 to 7 hours prior to administration of the MRI composition, such as from 0 to 1 hour, such as from 1 to 2 hours, such as from 2 to 3 hours, such as from 3 to 4 hours, such as from 4 to 5 hours, such as from 5 to 6 hours, such as from 6 to 7 hours prior to administration of the MRI composition.

10. The MRI composition for use according to any one of the preceding claims, wherein the subject has consumed the meal within about 1 hour prior to administration of the MRI composition, such as within about 45 minutes, such as within about 30 minutes prior to administration of the MRI composition.

11. The MRI composition for use according to any one of the preceding claims, wherein the meal comprises a predefined amount of carbohydrates, a predefined amount of fats, and a predefined amount of proteins.

12. The MRI composition for use according to any one of the preceding claims, wherein the meal is a snack.

13. The MRI composition for use according to any one of the preceding claims, wherein the predefined amount of carbohydrates in the meal is from 20 gram to 50 gram, such as from 20 to 22 gram, such as from 22 to 24 gram, such as from 24 to 26 gram, such as from 26 to 28 gram, such as from 28 to 30 gram, such as from 30 to 32 gram, such as from 32 to 34 gram, such as from 34 to 36 gram, such as from 36 to 38 gram, such as from 38 to 40 gram, such as from 40 to 42 gram, such as from 42 to 44 gram, such as from 44 to 46 gram, such as from 46 to 48 gram, such as from 48 to 50 gram.

14. The MRI composition for use according to any one of the preceding claims, wherein the predefined amount of carbohydrates in the meal is about 30 gram or about 31 gram.

15. The MRI composition for use according to any one of the preceding claims, wherein the predefined amount of carbohydrates in the meal is from 60 to 200 kcal, such as from 60 to 70 kcal, such as from 70 to 80 kcal, such as from 80 to 90 kcal, such as from 90 to 100 kcal, such as from 100 to 110 kcal, such as from 110 to 120 kcal, such as from 120 to 130 kcal, such as from 130 to 140 kcal, such as from 140 to 150 kcal, such as from 150 to 160 kcal, such as from 160 to 170 kcal, such as from 170 to 180 kcal, such as from 180 to 190 kcal, such as from 190 to 200 kcal.

16. The MRI composition for use according to any one of the preceding claims, wherein the predefined amount of carbohydrates in the meal is from 50 to 70 kcal % of the total amount of calories in the meal, such as from 50 to 52 kcal %, such as from 52 to 54 kcal %, such as from 54 to 56 kcal %, such as from 56 to 58 kcal %, such as from 58 to 60 kcal %, such as from 60 to 62 kcal %, such as from 62 to 64 kcal %, such as from 64 to 66 kcal %, such as from 66 to 68 kcal %, such as from 68 to 70 kcal % of the total amount of calories in the meal.

17. The MRI composition for use according to any one of the preceding claims, wherein the predefined amount of carbohydrates in the meal is about 59% of the total amount of calories in the meal.

18. The MRI composition for use according to any one of the preceding claims, wherein the predefined amount of fats in the meal is from 5 gram to 30 gram, such as from 5 to 10 gram, such as from 10 to 15 gram, such as from 15 to 20 gram, such as from 20 to 25 gram, such as from 25 to 30 gram.

19. The MRI composition for use according to any one of the preceding claims, wherein the predefined amount of fats in the meal is about 7 gram, about 8 gram, about 9 gram, or about 10 gram.

20. The MRI composition for use according to any one of the preceding claims, wherein the predefined amount of fats in the meal is from 30 to 150 kcal, such as from 30 to 40 kcal, such as from 40 to 50 kcal, such as from 50 to 60 kcal, such as from 60 to 70 kcal, such as from 70 to 80 kcal, such as from 80 to 90 kcal, such as from 90 to 100 kcal, such as from 100 to 110 kcal, such as from 110 to 120 kcal, such as from 120 to 130 kcal, such as from 130 to 140 kcal, such as from 140 to 150 kcal.

21. The MRI composition for use according to any one of the preceding claims, wherein the predefined amount of fats in the meal is from 25 to 45 kcal % of the total amount of calories in the meal, such as from 25 to 27 kcal %, such as from 27 to 29 kcal %, such as from 29 to 31 kcal %, such as from 31 to 33 kcal %, such as from 33 to 35 kcal %, such as from 35 to 37 kcal %, such as from 37 to 39 kcal %, such as from 39 to 41 kcal %, such as from 41 to 43 kcal %, such as from 43 to 45 kcal % of the total amount of calories in the meal.

22. The MRI composition for use according to any one of the preceding claims, wherein the predefined amount of fats in the meal is about 36% of the total amount of calories in the meal.

23. The MRI composition for use according to any one of the preceding claims, wherein the predefined amount of proteins in the meal is from 0 gram to 10 gram, such as from 0 to 2 gram, such as from 2 to 4 gram, such as from 4 to 6 gram, such as from 6 to 8 gram, such as from 8 to 10 gram.

24. The MRI composition for use according to any one of the preceding claims, wherein the predefined amount of proteins in the meal is about 0 gram, about 1 gram, about 2 gram, or about 3 gram.

25. The MRI composition for use according to any one of the preceding claims, wherein the predefined amount of proteins in the meal is from 0 to 50 kcal, such as from 0 to 5 kcal, such as from 5 to 10 kcal, such as from 10 to 15 kcal, such as from 15 to 20 kcal, such as from 20 to 25 kcal, such as from 25 to 30 kcal, such as from 30 to 35 kcal, such as from 35 to 40 kcal, such as from 40 to 45 kcal, such as from 45 to 50 kcal.

26. The MRI composition for use according to any one of the preceding claims, wherein the predefined amount of proteins in the meal is from 0 to 10 kcal % of the total amount of calories in the meal, such as from 0 to 1 kcal %, such as from 1 to 2 kcal %, such as from 2 to 3 kcal %, such as from 3 to 4 kcal %, such as from 4 to 5 kcal %, such as from 5 to 6 kcal %, such as from 6 to 7 kcal %, such as from 7 to 8 kcal %, such as from 8 to 9 kcal %, such as from 9 to 10 kcal % of the total amount of calories in the meal.

27. The MRI composition for use according to any one of the preceding claims, wherein the predefined amount of proteins in the meal is about 5% of the total amount of calories in the meal.

28. The MRI composition for use according to any one of the preceding claims, wherein the meal comprises one or more food items selected from the group consisting of: bread, such as toast bread, and fruit juice, such as lime juice.

29. The MRI composition for use according to any one of the preceding claims, wherein the juice is provided at a volume of from 100 mL to 400 mL, such as from 100 mL to 110 mL, such as from 110 mL to 120 mL, such as from 120 mL to 130 mL, such as from 130 mL to 140 mL, such as from 140 mL to 150 mL, such as from 150 mL to 160 mL, such as from 160 mL to 170 mL, such as from 170 mL to 180 mL, such as from 180 mL to 190 mL, such as from 190 mL to 200 mL, such as from 200 mL to 210 mL, such as from 210 mL to 220 mL, such as from 220 mL to 230 mL, such as from 230 mL to 240 mL, such as from 240 mL to 250 mL, such as from 250 ml to 260 mL, such as from 260 mL to 270 mL, such as from 270 mL to 280 mL, such as from 280 mL to 290 mL, such as from 290 mL to 300 mL, such as from 300 mL to 310 mL, such as from 310 mL to 320 mL, such as from 320 mL to 330 mL, such as from 330 mL to 340 mL, such as from 340 mL to 350 mL, such as from 350 mL to 360 mL, such as from 360 mL to 370 mL, such as from 370 mL to 380 mL, such as from 380 mL to 390 mL, such as from 390 mL to 400 mL.

30. The MRI composition for use according to any one of the preceding claims, wherein the calorie content of the meal is from 100 kcal to 500 kcal, such as from 100 to 110 kcal, such as from 110 kcal to 120 kcal, such as from 120 kcal to 130 kcal, such as from 130 kcal to 140 kcal, such as from 140 kcal to 150 kcal, such as from 150 kcal to 160 kcal, such as from 160 kcal to 170 kcal, such as from 170 kcal to 180 kcal, such as from 180 kcal to 190 kcal, such as from 190 kcal to 200 kcal, such as from 200 kcal to 210 kcal, such as from 210 kcal to 220 kcal, such as from 220 kcal to 230 kcal, such as from 230 kcal to 240 kcal, such as from 240 kcal to 250 kcal, such as from 250 kcal to 260 kcal, such as from 260 kcal to 270 kcal, such as 270 kcal to 280 kcal, such as from 280 kcal to 290 kcal, such as from 290 kcal to 300 kcal, such as from 300 kcal to 310 kcal, such as from 310 kcal to 320 kcal, such as from 320 kcal to 330 kcal, such as from 330 kcal to 340 kcal, such as from 340 kcal to 350 kcal, such as from 350 kcal to 360 kcal, such as from 360 kcal to 370 kcal, such as from 370 kcal to 380 kcal, such as from 380 kcal to 390 kcal, such as from 390 kcal to 400 kcal.

31. The MRI composition for use according to any one of the preceding claims, wherein the calorie content of the meal is about 300 kcal or below, and wherein the predefined amount of carbohydrates in the meal constitutes at least 50% of the calorie content of the meal.

32. A method for Magnetic Resonance Imaging (MRI) of a subject comprising, administering a composition comprising separately or together, manganese (II) chloride tetrahydrate, L-alanine and a vitamin D, and optionally one or more water-soluble excipients to the subject; and performing MRI on said subject; wherein the subject has consumed a meal from 0 to 8 hours prior to or up to 2 hours subsequent to administration of the MRI composition, and wherein the calorie content of the meal is about 500 kcal or below.

33. A method of reducing or preventing nausea, diarrhea, and/or headaches in a subject receiving an MRI composition comprising, separately or together, manganese (II) chloride tetrahydrate, L-alanine and a vitamin D, and optionally one or more water-soluble excipients; the method comprising:
i) administering a meal to the subject; and
ii) administering the MRI composition to the subject;
wherein the meal is administered from 0 to 8 hours prior to or up to 2 hours subsequent to administering the MRI composition.

34. The method according to claim 33, wherein the meal is administered from 0 to 6 hours prior to the administering of the MRI composition, such as from 0 to 1 hour, such as from 1 to 2 hours, such as from 2 to 3 hours, such as from 3 to 4 hours, such as from 4 to 5 hours, such as from 5 to 6 hours prior to the administering of the MRI composition.

35. The method according to any one of claims 32 to 34, wherein the imaging quality is unaffected compared to a method where the MRI composition is administered to a fasting subject, wherein the fasting subject has not been eating for more than 8 hours.
